(19) United States
(12) Patent Application Publication
(10) Pub. No.: US 2003/0220689 A1
(43) Pub. Date: Nov. 27, 2003

(54) Device and method for assisting in positioning implants

(76) Inventors: Stephen Ritland, Flagstaff, AZ (US); Mario Zeiss, Poing (DE); Matthias Brundobler, Munchen (DE)

Correspondence Address: RENNER, OTTO, BOISSELLE & SKLAR, LLP, Nineteenth Floor, 1621 Euclid Avenue, Cleveland, OH, 44115-2191, US

(21) Appl. No.: 10/395,019

(22) Filed: Mar. 21, 2003

(30) Foreign Application Priority Data
Mar. 21, 2002 (EP) 02 006 574.4

(51) Int. Cl. A61F 2/28

(52) U.S. Cl. 623/16.11

(57) ABSTRACT

A device for assisting in positioning implants and/or implanting aids on or in a bone structure includes an outer part having a holder section and a guide section, and an inner part having a guide engagement section and a passage defined within the guide engagement section for an implant and/or implanting aid. A navigational reference for navigating the device by means of a medical navigation system can be on the outer part and/or on the inner part, by which the implant and/or implanting aid can be positioned with navigational assistance.

### FIELD OF THE INVENTION

The present invention relates to a device for assisting in positioning implants and/or implanting aids on or in a bone structure, such as on a vertebra. Such devices are also called “retractors” and serve, for example, to insert pedicle screws in a minimally invasive manner.

### BACKGROUND OF THE INVENTION

The most common method for positioning implants and/or implanting aids is to expose the entire area above the relevant bone structure, for example, above a vertebra, with the aid of a large cut into the skin and then to insert the implants to be inserted, for example, screws, via threaded drill holes to be cut beforehand. The operating surgeon must have great experience in this respect. Physicians with little experience could drill into structures, which absolutely must be left intact, for example, the spinal channel of a vertebra. An additional disadvantage is that such surgery is maximally invasive. Mechanical insertion aids intended to enable less invasive surgery, for example, arrangements of pivoting arms, are devices, which are exceedingly mechanically complicated to construct and also can only be handled correctly with great experience.

Where drilling or insertion aids, which include a drill hole or passage via which implants or implanting aids are to be positioned in a directed manner onto the bone structure, are used, one conventional method of positioning includes using navigation aids, such as known navigational pointers. These navigation aids, which can be inserted into such a drill hole, are supposed to determine the orientation of the implant or implanting aid in the navigation system. However, these methods have proven to be insufficient because considerable angular deviations can arise due to the relatively large drill hole. Because of the necessary accuracy in such surgery, these methods are fundamentally not suitable. One-piece emitter-navigated drill guides are, for example, known from WO 96/11624 and U.S. Pat. No. 5,517,990.

### SUMMARY OF THE INVENTION

One object of the present invention is to provide a device for assisting in positioning implants and/or implanting aids, which allows minimally invasive surgery with a high degree of accuracy. This object can be achieved in accordance with the invention by a device for assisting in positioning implants and/or implanting aids on or in a bone structure, in particular on a vertebra. The device can include an outer part including a holder section and a guide section and an inner part comprising a guide engagement section and a passage for an implant and/or implanting aid. A reference means for navigating the device using a medical navigation system can be arranged on the outer part and/or on the inner part, by which the implant and/or implanting aid can be positioned with navigational assistance.

Within the context of the present description, the terms “inner part” and “outer part” do not necessarily serve to define an inner-outer positional relationship of these parts. They are merely intended to indicate that the “outer part” is the part of the device serving as a guide, in which the “inner part” can be moved and positioned in a particular direction.

Medical navigation systems such as may in principle be used within the framework of the present invention are known for example from DE 196 39 615 C2 or from U.S. Pat. No. 6,285,902, which are incorporated herein by reference in their entirety. Since a device in accordance with the invention can be navigated in the medical navigation system by means of its reference means, implants can be positioned or implanting aids used very precisely and purposefully as well as controllably using the device. This, in turn, makes it possible to perform the corresponding surgery in a minimally invasive manner. Traumas to the soft tissue preceding the bone to be treated can be reduced to a minimum. It is no longer necessary to use other, possibly inaccurate aids, such as mechanical insertion aids or navigational pointers. In addition, a surgeon can plan the point of entry, angle of entry and the size of implants or implanting aids sufficiently accurately in advance, so that undesirable damage to parts of the patient's body can be avoided.

In one embodiment, real time tracking of the device is possible, which then can only require and use the fluoroscopy images, which often were used and which are recorded in the interim, as a supplementary navigational aid. Fluoroscopy, which is relatively unsuitable as a sole navigational instrument, since new x-ray images constantly have to be produced, then becomes a suitable supplementary instrument for navigation, in order, for example, to make bone structures more easily visible in the tomographic images of the navigation system, as is, for example, described in EP 1 153 572 A1, which is incorporated herein by reference in its entirety.

When implants and/or implanting aids are mentioned within the context of the present invention, these terms include both structures remaining in the body permanently or for a long time as well as those that are removed again directly after being inserted. In addition, implants and/or implanting aids include aids that are used for introducing drill holes and channels into a bone structure and are then removed again. With the aid of the device in accordance with the invention, it is possible to introduce tools, implants, but also substances, such as liquids (e.g., bone cement).

In accordance with an embodiment of the present invention, the reference means of the device is arranged on the inner part. The inner part can then constantly remain navigationally monitored, even when it is separated from the outer part after the positioning process, such as, for example, when the outer part is withdrawn from the inner part once the latter has been pre-positioned. This enables the surgeon to use a number of non-navigated outer parts. In this way, identical method steps can be performed on different parts of the body beforehand. For example, a number of outer parts can be put in position in order to then enable a navigated inner part to respectively access its positioning point. The inner part is then removed from the one outer part and inserted into the next for, respectively, the same method step.

In another embodiment of the present invention, the reference means of the device can be arranged on the outer part. Such an arrangement is especially advantageous when the outer part only has to be positioned together with the inner part once and all of the surgery to be performed with the aid of the device can be completed successively at one point.

It is equally possible in accordance with the present invention to arrange a reference means both on the outer part and on the inner part. There then exists, for example, the option of firstly inserting an outer part up to the desired point of treatment and thus performing advance positioning for the subsequent insertion of the inner part. Navigation can then be continued based on the navigated inner part for as long as the actual treatment lasts, since the inner part can be arranged exactly on the predetermined positioning point of the bone structure. If distinctive reference means are respectively used for the outer part and inner part, then distinguishing these two parts of the device when navigating poses no problem.

In one embodiment, a number of inner parts are provided with the device. The inner parts can include passages of different sizes for implants and/or implanting aids having different dimensions. In this way, it is possible to guide the instruments respectively used, (for example, milling device, threading device, implant), through the respective passage of the inner part being used, precisely and with an exact external fitting.

A positioning tip can be provided on the inner part and/or outer part. The shape of the positioning tip can be adapted such that it can be inserted in a defined position into a matching calibration receptacle of a calibration instrument. Such a calibration instrument serves to communicate to the navigation system the exact position and orientation of the device at a point in time before it is used. This is important, for example, when removable reference means are used, which can be positioned on devices of any embodiment. If devices with standardized inner and outer parts are used, it is sufficient to communicate the type of device to the navigation system beforehand, which removes the necessity for calibrating.

In order to make the inner part easy to handle, a grip section can be arranged on it.

According to its application, the device in accordance with the invention can be made of various materials. The inner part and the outer part can be made of, for example, a metal, such as steel (e.g., surgical steel). If a metal, such as steel, is used to produce the parts of the device, a reference means that may be tracked using an optically based navigation system is suitable as the reference means. The reference means can include an arrangement of reflectors or emitters.

In another embodiment, there exists the possibility of producing the inner part and/or the outer part from plastic, such as, for example, Kevlar. This can be advantageous when the reference means is one which may be tracked using a magnetically based navigation system, such as an arrangement of miniature coils. It follows from the above that the device in accordance with the invention does in principle use one navigation system, but that the user has a relatively free choice of navigation system.

In accordance with another preferred embodiment of the present invention, the inner part and/or the outer part can be made of a material which is permeable to x-rays. This can ensure that the instruments do not throw any visible shadows on x-rays images produced during treatment, which could obscure parts of the structures which ought to be made visible.

### BRIEF DESCRIPTION OF THE DRAWINGS

These and further features of the present invention will be apparent with reference to the following description and drawings, wherein:

FIG. 1 is a perspective view of a device for positioning implants and/or implanting aids in accordance with the present invention; and

FIG. 2 is a perspective view of a device for positioning implants and/or implanting aids along with an associated section of vertebrae in accordance with an alternative embodiment of the present invention.

### DETAILED DESCRIPTION OF THE INVENTION

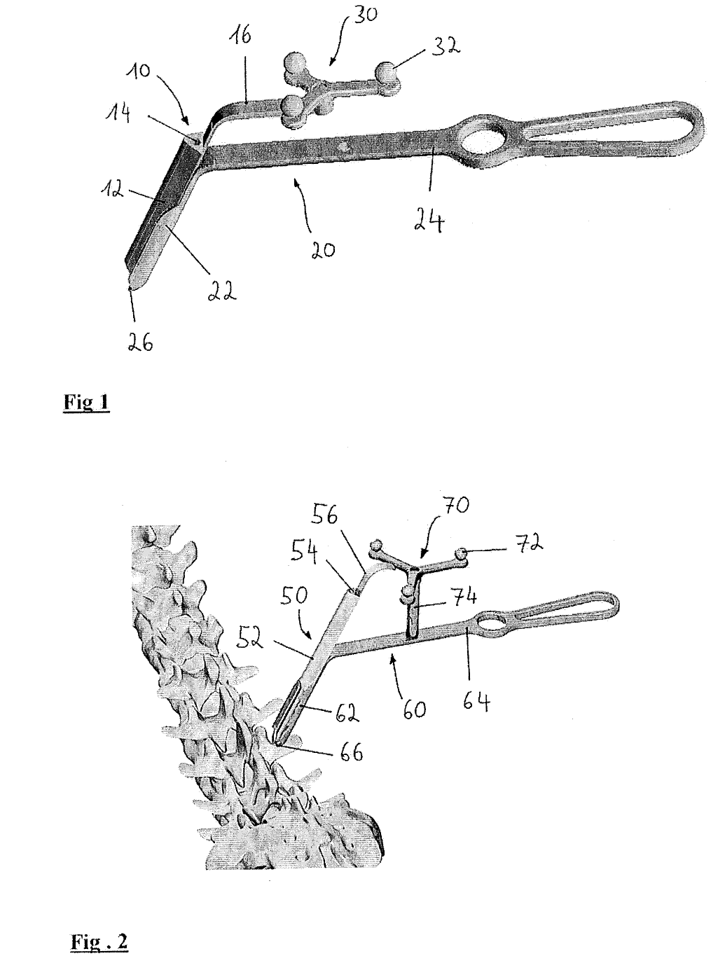

With reference to FIG. 1, a device for positioning implants and/or implanting aids (also referred to as a retractor) includes an inner part 10 and an outer part 20. Within the context of the present description, the terms “inner part” and “outer part” do not necessarily serve to define an inner-outer positional relationship of these parts. They are merely intended to indicate that the “outer part” is the part of the device serving as a guide, in which the “inner part” can be moved and positioned in a particular direction. The inner part 10 includes a guide engagement section 12, which is guided in a guide section 22 of the outer part 20. In one embodiment, the guide section 22 forms a sort of rail for the guide engagement section 12, which receives a lower part of the triangular guide engagement section 12 in the manner of a longitudinal guide. While the guide engagement section 12 is illustrated as having a triangular cross-section, it is to be appreciated that other cross-sectional geometries may be employed without departing from the scope of the present invention.

The guide engagement section 12 can include a continuous longitudinal drill hole or passage 14, through which instruments, such as mills or threading means and, for example, implants can be inserted and guided into a bone structure. In one embodiment, a reference means 30 (also referred to as a navigational star) is attached to the inner part 10 and/or to the guide engagement section 12 via an extension 16, which can also be used as a grip piece for the inner part 10. In one embodiment, the reference means 30 includes an arrangement of three reflectors 32 whose position can be detected by a navigation system. The reflectors 32 of the navigational star 30 can have an arrangement which is distinctive for the inner part 10 and thus allow its position to be tracked at any point in time. One exemplary navigation system for use with the present invention is described in commonly assigned U.S. Pat. No. 6,351,659, which is incorporated herein by reference in its entirety.

As discussed above, the guide engagement section 12 of the inner part 10 can be guided in the guide section 22 of the outer part 20 in a rail-like manner. The outer part 20 can include a positioning tip 26 at the front end of the guide section 22, with which tip 26 the outer part 20 can be exactly positioned onto a predetermined positioning point on the bone structure. In an alternative embodiment, such a positioning tip 26 can also be provided on the inner part 10, i.e. on its front tip.

A holder section or handle 24 is connected to the guiding section 22, such that the surgeon can grip the holder section 24 optimally, when using the device.

FIG. 2 illustrates an alternative embodiment of the device in which the reference means (also referred to as the reference star) 70 is not attached to the inner part 50, but to the outer part 60, namely, to the holder section or handle 64 thereof. This can be achieved using a fixing rod 74, which bears the reference star 70, including its associated reflectors 72, at its upper end.

The outer part 60 of the device can include a holder section or handle 64, a guide section 62 and a positioning tip 66. The inner part 50 can include a guide engagement section 52 including a passage 54, i.e. a drilled-through hole, extending longitudinally through the guide engagement section 52. In this case, the extension 56 connected to the guide engagement section 52 is provided as a grip piece by which the inner part 50 can be handled. FIG. 2 shows how the device in accordance with the second embodiment of the invention can be positioned on a vertebra.

In order to explain the invention further, the application of the device within the context of an operation will now be described in the following, in which operation pedicle screws are inserted into vertebrae and connected on the outside with rods.

First, a short cut into the skin can be made centrally above the vertebrae to be treated, where the cut only needs to be a few centimeters long, which is much shorter than in conventional “open operations”. The cut can be deepened as far as the muscle packing and then the skin surrounding the muscle packing can be opened. The pedicle, i.e. the area into which the screw is to be inserted, can be probed and the device in accordance with the invention, with its outer part 20, 60 and inner part 10, 50, can be inserted between the muscle packing, until the positioning tip 26, 66 lies exactly at the position where the screws are to enter. Then the position of penetration can be checked and corrected, as appropriate, using the navigation system, and the trajectory of penetration is planned. Because the reflector 32, 72 in accordance with the invention can be navigated via the reference means 30, 70, the trajectory can, for example, be calculated in the navigation system from various angles and displayed on a screen. The position of the retractor can be corrected by orientating the outer part 20, 60, until a suitable angle of penetration is secured.

The bone can then be opened at the point of entry using, for example, a mill, through the passage or bore 14, 54 in the guide engagement section 12, 52 of the inner part 10, 50. The mill is then removed again from the passage 14, 54 of the inner part 10, 50 and a threader is then inserted, with the aid of which the thread is introduced in the pedicle.

Once the threader has been removed, a pedicle probe can be used to verify whether or not the pedicle has been perforated up to the inner wall, which, given suitable navigation, should never occur.

The inner part 10, 50 of the device in accordance with the invention can then be removed and the screw can be inserted in along the prepared thread on the guide section of the outer part.

This process can be repeated for a number of screws on vertebrae lying one above the other, which need not be adjacent to each other, until a sufficient number of screws has been inserted, which are then connected to rods. As already described above, there of course also exists the possibility of altering the sequence described above. For example, a number of identical method steps can be performed in immediate succession on different vertebrae, after which a second group of method steps follows. Setting liquid implants (bone graft) can of course be introduced in this way, where the device in accordance with the invention can then be used as a guide for a cannula.

Although particular embodiments of the invention have been described in detail, it is understood that the invention is not limited correspondingly in scope, but includes all changes, modifications and equivalents coming within the spirit and terms of the claims appended hereto.

European Patent Application No. 02 006 574.4 is hereby incorporated herein by reference in its entirety. A certified copy of said European patent application is appended hereto and forms a part of this nonprovisional application.

What is claimed is:

1. A device for assisting in positioning implants or implanting aids on or in a bone structure, said device comprising:
an outer part including a holder section and a guide section (22, 62);
an inner part including a guide engagement section defining a passage for receiving implant or implanting aid;
a navigational reference for navigating the device using a medical navigation system, said navigational reference being disposed on at least one of (i) the inner part and (ii) the outer part.

2. The device as set forth in claim 1, wherein the implant or implanting aid is positioned with navigational assistance.

3. The device as set forth in claim 1, wherein the navigational reference is disposed on the inner part.

4. The device as set forth in claim 1, wherein the navigational reference is disposed on the outer part.

5. The device as set forth in claim 1, wherein a navigational reference is disposed on the outer part and on the inner part.

6. The device as set forth in claim 1, further comprising:
a plurality of inner parts, said plurality of inner parts defining passages of different sizes for receiving implants or implanting aids having different dimensions.

7. The device as set forth in claim 1, further comprising:
a positioning tip disposed on at least one of (i) the inner part and (ii) the outer part, said positioning tip having a shape such that it can be inserted in a defined position into a matching calibration receptacle of a calibration instrument.

8. The device as set forth in claim 1, further comprising:
a grip piece connected to the inner part.

9. The device as set forth in claim 1, wherein at least one of (i) the inner part and (ii) the outer part is comprised of metal.

10. The device as set forth in claim 9, wherein at least one of (i) the inner part and (ii) the outer part is comprised of steel.

11. The device as set forth in claim 9, wherein the navigational reference includes an arrangement of reflectors or emitters, said navigational reference being operative to be tracked using an optically based navigation system.

12. The device as set forth in claim 1, wherein at least one of (i) the inner part and (ii) the outer part is comprised of plastic.

13. The device as set forth in claim 1, wherein at least one of (i) the inner part and (ii) the outer part is comprised of Kevlar.

14. The device as set forth in claim 12, wherein the navigational reference includes an arrangement of miniature coils, said navigational reference being operative to be tracked using a magnetically based navigation system.

15. The device as set forth in claim 13, wherein at least one of (i) the inner part and (ii) the outer part is made of a material which is permeable to x-rays.
